(19) United States
(12) Patent Application Publication
(10) Pub. No.: US 2003/0077692 A1
(43) Pub. Date: Apr. 24, 2003

(54) REFOLDING METHOD

(76) Inventors: ALAN ROY FERSHT, CAMBRIDGE (GB); MYRIAM MARLENNE ALTAMIRANO, CAMBRIDGE (GB)

Correspondence Address: FROMMER LAWRENCE & HAUG, 745 FIFTH AVENUE- 10TH FL., NEW YORK, NY, 10151, US

(21) Appl. No.: 09/415,849

(22) Filed: Oct. 7, 1999

Related U.S. Application Data
(63) Continuation-in-part of application No. PCT/GB98/02218, filed on Jul. 24, 1998.

(30) Foreign Application Priority Data
Jul. 24, 1997 (GB) 9715634.3
Aug. 28, 1997 (GB) 9718259.6
Jul. 2, 1998 (GB) 9814314.2

(51) Int. Cl. C12P 21/06

(52) U.S. Cl. 435/68.1

(57) ABSTRACT

The invention relates to a method for promoting the folding of a polypeptide, comprising the step of contacting the polypeptide with a molecular chaperone and a foldase.

### FIELD OF THE INVENTION

The present invention relates to a method for refolding polypeptides, particularly insoluble or misfolded polypeptides, using a combination of a minichaperone peptide and a foldase wherein the minichaperone and/or foldase are imnmobilised on a solid support. The present invention also relates to a refolding matrix comprising a minichaperone peptide and a protein disulphide isomerase immobilised thereon.

### BACKGROUND TO THE INVENTION

Many proteins, especially those that are secreted by eukaryotes, are stabilised by disulphide bonds. Examples of such proteins include those used for medical or biotechnological use, such as interleukins, interferons, antibodies and their fragments, insulin, transforming growth factor, as well as many toxins and proteases. The folding of disulphide-containing proteins is often slow in vitro and coupled with the acquisition of the native chain conformation. Even under optimal conditions, the uncatalysed oxidative refolding of reduced ribonuclease (RNase) has a half-life of about 1.5 h and bovine pancreatic trypsin inhibitor (BPTI) refolds even more slowly (t_{½}˜8 h). Further, there is a usually a mixture of products, containing various combinations of correctly and incorrectly formed bonds. The refolding of many desirable proteins is often very difficult in vitro because the unwanted products cause greatly lowered yields and contaminants.

Chaperones are in general known to be large multisubunit protein assemblies essential in mediating polypeptide chain folding in a variety of cellular compartments. Families of chaperones have been identified, for example the chaperonin hsp60 family otherwise known as the cpn60 class of proteins are expressed constitutively and there are examples to be found in the bacterial cytoplasm (GroEL), in endosymbiotically derived mitochondria (hsp60) and in chloroplasts (Rubisco binding protein). Another chaperone family is designated TF55/TCP1 and found in the thermophilic archaea and the evolutionarily connected eukaryotic cytosol. A comparison of amino acid sequence data has shown that there is at least 50% sequence identity between chaperones found in prokaryotes, mitochondria and chloroplasts (Ellis R J and Van der Vies S M (1991) Ann Rev Biochem 60: 321-347).

A typical chaperonin is GroEL which is a member of the hsp60 family of heat shock proteins. GroEL is a tetradecamer wherein each monomeric subunit (cpn60 m) has a molecular weight of approximately 57 kD. The tetradecamer facilitates the in vitro folding of a number of proteins which would otherwise misfold or aggregate and precipitate. The structure of GroEL from E. coli has been established through X-ray crystallographic studies as reported by Braig K et al (1994) Nature 371: 578-586. The holo protein is cylindrical, consisting of two seven-membered rings that form a large central cavity.

The entire amino acid sequence of E. coli GroEL is also known (see Braig K et al (1994) supra) and three domains have been ascribed to each cpn60 m of the holo chaperonin (tetradecamer). These are the intermediate (amino acid residues 1-5, 134-190, 377-408 and 524-548), equatorial (residues 6-133 and 409-523) and apical (residues 191-376) domains.

GroEL facilitates the folding of a number of proteins by two mechanisms; (1) it prevents aggregation by binding to partly folded proteins (Goloubinoff P et al (1989) Nature 342: 884-889; Zahn R and Plückthun A (1992) Biochemistry 31: 3249-3255), which then refold on GroEL to a native-like state (Zahn R and Plückthun A (1992) Biochemistry 31: 3249-3255; Gray T E and Fersht A R (1993) J Mol Biol 232: 1197-1207); and (2) it continuously anneals misfolded proteins by unfolding them to a state from which refolding can start again (Zahn R et al (1996) Science 271: 642-645).

Yoshida et al (1993) FEBS 336: 363-367 report that a 34kD proteolytic fragment of E. coli GroEL which lacks 149 NH₂-terminal residues and ˜93 COOH-terminal residues (GroEL 150-456) facilitates refolding of denatured rhodanese in the absence of GroES and ATP. Although the proteolytic fragment GroEL 150-456 elutes as a monomer during gel filtration, it still comprises the apical domain and significant portions of the intermediate and equatorial domains, the latter of which determine the intersubunit contacts of GroEL (Braig K et al (1994) supra), thus allowing transient formation of the central cavity thereby accounting for the chaperonin activity which is observed.

Taguchi H et al (1994) J Biol Chem 269: 8529-8534 report that a transiently formed GroEL tetradecamer (the holo-chaperonin) was perceived to exist when the chaperonin monomers are present in solution. Consequently, the refolding activity of these preparations can be seen to be caused by the presence of holo chaperonin, not monomers. To test this, Taguchi et al immobilised cpn60 m to a chromatographic resin to exclude the possibility of holo chaperonin formation. When immobilised and therefore when in truly monomeric form, cpn60 m exhibited only about 10% rhodanese refolding activity.

Alconada A and Cuezva J M (1993) TIBS 18: 81-82 suggested that an “internal fragment” of GroEL may possess a chaperone activity on the basis of amino acid sequence similarity between the altered mRNA stability (ams) gene product (Ams) of E. coli and the central part of GroEL. The ams locus is a temperature-sensitive mutation that maps at 23 min on the E. coli chromosome and results in mRNA with an increased half-life. The ams gene has been cloned, expressed and shown to complement the ams mutation. The gene product is a 149-amino acid protein (Ams) with an apparent molecular weight of 17 kD.

Chanda P K et al (1985) J Bacteriol 161: 446-449 found that a 17 kD protein fragment corresponding to part of the L gene of the groE operon, when expressed in E. coli ams mutants restores the wild-type phenotype. This 17 kD fragment was suggested as being an isolated, functional chaperonin protein module. The amino acid sequences of three chaperonins (E. coli GroEL, ribulose bisphosphate carboxylase (RUBPC) subunit-binding protein from Triticum aestivum and Saccharomyces cerevisiae mitochondrial hsp60) were compared with the sequence of Ams. Residues 307-423 were found to correspond substantially between Ams and GroEL. These residues comprise nearly equivalent portions of both the intermediate and apical domains of GroEL.

More recently, experiments have been designed with the aim of dissecting out the active site of GroEL and examining its activity in isolation from the tetradecameric structure of the intact GroEL protein (Zahn, et al., (1996) PNAS(USA) 93:15024-15029; Buckle et al., (1997) PNAS(USA) 94:3571-3579). Functionally active monomeric minichaperones have been produced, which are active in solution (Zahn et al., Supra) or immobilised on a solid support (Altamirano et al., (1997) PNAS(USA) 94:3576-3578). Minichaperone proteins which are active in refolding misfolded or unfolded polypeptides are described in our copending international patent application PCT/GB96/02980, filed on Dec. 3, 1996, and UK patent application 9620243.7, filed Sep. 26, 1996.

Minichaperones (e.g. a peptide consisting of residues 191-345; or 191-376, or smaller fragments of GroEL) that are immobilised on agarose have very efficient chaperoning activity with several proteins. Refolding chromatography can be performed using column chromatography or, more conveniently, by batchwise shaking of reagents.

In addition to molecular chaperones, the complex protein folding machinery in the cell comprises thiol/disulphide oxidoreductases, such as protein disulphide isomerase (PDI). In vivo, disulphide bond formation is catalysed by PDI in the endoplasmic reticulum of eukaryotes and by DsbA protein in the periplasm of bacteria (Goldberger et al., (1963) J. Biol. Chem. 238:628-635; Zapun, et al., (1992) Proteins 14, 10-15). These also catalyse the shuffling of incorrectly formed disulphide bonds. PDI is a very abundant protein; the concentration in the endoplasmic reticulum lumen has been estimated to be near-millimolar (Lyles, M. and Gilbert, H. (1991) Biochemistry 30:619-625). A high local concentration along with high chemical reactivity as an oxidant favours a rapid second-order reaction with unfolded substrates, making oxidation competitive with initial folding.

Thiol/disulphide oxidoreductases are known from a variety of species and have been proposed for use in refolding recombinantly produced polypeptides.

WO94/08012 (Research Corp. technologies, Inc.) discloses the coexpression of a thiol/disulphide oxidoreductase (PDI) with a recombinantly produced polypeptide and optionally with a molecular chaperone (BiP) in order to facilitate refolding. However, no teaching is provided concerning the possible use of minichaperones with PDI, or of refolding possibilities other than coexpression. Moreover, no data or conclusions concerning the possible utility of such a combination are disclosed.

WO94/02502 (Genetics Institute, Inc.) discloses the expression of fusion polypeptides with thioredoxins, such as the thioredoxin-like domain of PDI, which increases the yield of soluble, stable polypeptide. However, the combination of molecular chaperones and PDIs is not discussed.

Morjana, N. and Gilbert, H. (1994) Protein Expression and Purification 5:144-148 immobilised bovine liver PDI on CNBr-activated agarose and, using columns containing 4.5 mg of protein per mL of gel, obtained a yield of 55% active RNase A from its oxidised and disulphide-scrambled denatured state. At a lower concentration of PDI (1 mg per mL of gel), the yield of refolded RNase from scrambled RNase rose to 89 per cent. In all cases batch mode activity was not obtained. This is paradoxical, not only because of the apparent higher activity at lower PDI concentrations, but also because the presence of activity in both batchwise and chromatographic experiments is a test of whether the supposed activity is associated with the immobilised reagent. The lack of activity in batch mode shows that it is unlikely that the activity in the column chromatography results from the immobilised material, but is possibly an artefact of leakage from CNBr-activated agarose. Moreover, the combination of PDIs and a molecular chaperone is not suggested.

The refolding machinery also comprises peptidyl prolyl cis-trans isomerase (PPI). PPIs catalyse the cis-trans isomerisation of peptidyl-prolyl bonds (Schmid et al. (1993) Accessoiy Folding Proteins, 25-65. Academic Press, Inc, New York). The peptide bond is overwhelmingly in the trans conformation in native and denatured peptides apart from the peptidyl-prolyl bond, which is predominantly trans in denatured states but can be in the cis conformation in folded proteins. PPIs appear to have a much smaller effect on the observed rate of protein folding than either chaperonins or PDIs (Freedman, (1992) Protein Folding. Freeman, N.Y.; Lorimer, (1993) Accessory Folding Proteins. Academic Press, Inc., New York).

### SUMMARY OF THE INVENTION

According to a first aspect of the present invention, there is provided a method for promoting the folding of a polypeptide comprising contacting the polypeptide with a molecular chaperone and a foldase wherein the foldase and optionally the molecular chaperone are immobilised onto a solid phase support.

The polypeptide is preferably an unfolded or misfolded polypeptide, and advantageously comprises a disulphide. The molecular chaperone is a preferably fragment of a molecular chaperone, preferably a fragment of any hsp-60 chaperone, and may be selected from the group consisting of mammalian hsp-60 and GroEL, or a derivative thereof.

In the case that the fragment is a fragment of GroEL, it advantageously does not have an Alanine residue at position 262 and/or an Isoleucine residue at position 267 of the sequence of intact GroEL. Preferably, it has a Leucine residue at position 262 and/or a Methionine residue at position 267 of the sequence of intact GroEL. The invention therefore encompasses the use of a fragment of GroEL comprising a Leucine residue at position 262 and/or a Methionine residue at position 267 of the sequence of intact GroEL for promoting the folding of a polypeptide.

In a preferred embodiment, the molecular chaperone fragment comprises a region which is homologous to at least one of fragments 191-376, 191-345 and 191-335 of the sequence of intact GroEL.

Advantageously, the foldase is selected from the group consisting of thiol/disulphide oxidoreductases and peptidyl prolyl isomerases.

Preferably, the thiol/disulphide oxidoreductase is selected from the group consisting of E. coli DsbA and mammalian PDI, or a derivative thereof. Preferably, the peptidyl prolyl isomerase is a cyclophilin.

The invention moreover concerns a method as described above wherein the molecular chaperone fragment and/or the foldase is immobilised onto a solid phase support, which may be agarose. Accordingly, the invention also provides a solid phase support having immobilised thereon a molecular chaperone fragment and/or a foldase, a column packed at least in part with such a solid phase support and a method for immobilising disulphide-containing polypeptides on a solid phase support. Preferably, the method comprises the steps of:

a) reducing the disulphide in the polypeptide with a reducing agent, and removing the reducing agent under conditions so as to prevent re-oxidation;

b) reversibly blocking the thiol groups of the polypeptide;

c) contacting the solid phase with the thiol-blocked polypeptide at a non-acidic pH;

d) blocking any remaining active groups and removing uncoupled polypeptide by washing; and

e) regenerating the thiol groups on the bound polypeptide.

In a further aspect, the present invention provides a composition comprising a combination of a molecular chaperone fragment and a foldase, optionally together with a diluent, carrier or excipient.

The results presented herein show that using the refolding matrix and refolding method of the present invention, it is possible to obtain a refolded polypeptide having at least 100% of the activity of the corresponding native protein, in some case greater than 100% of the activity of the corresponding native protein. Thus, in another aspect, the present invention provides a polypeptide obtained by the method of the invention. Preferably said polypeptide has at least 100% of the activity of the corresponding native protein, more preferably greater than 100%.

### BRIEF DESCRIPTION OF THE FIGURE

FIG. 1 is a flow sheet representing a method for a disulphide-containing peptide to a solid support.

### DETAILED DESCRIPTION OF THE INVENTION

Definitions

Polypeptide. As used herein, a polypeptide is a molecule comprising at lest one peptide bond linking two amino acids. This term is synonymous with “protein” and “peptide”, both of which are used in the art to describe such molecules. A polypeptide may comprise other, non-amino acid components. The polypeptide the folding of which is promoted by the method of the invention may be any polypeptide. Preferably, however, it is an unfolded or misfolded polypeptide which is in need of folding. Alternatively, however, it may be a folded polypeptide which is to be maintained in a folded state (see below).

Preferably, the polypeptide contains at least one disulphide. Such polypeptides may be referred to herein as disulphide-containing polypeptides.

Examples of polypeptides include those used for medical or biotechnological use, such as interleukins, interferons, antibodies and their fragments, insulin, transforming growth factor, and many toxins and proteases, as well as molecular chaperones, peptidyl-prolyl isomerases and thiol/disulphide oxidoreductases.

Promoting the folding. The invention envisages at least two situations. A first situation is one in which the polypeptide to be folded is in an unfolded or misfolded state, or both. In this case, its correct folding is promoted by the method of the invention. A second situation is one in which the polypeptide is substantially already in its correctly folded state, that is all or most of it is folded correctly or nearly correctly. In this case, the method of the invention serves to maintain the folded state of the polypeptide by affecting the folded/unfolded equilibrium so as to favour the folded state. This prevents loss of activity of an already substantially correctly folded polypeptide. These, and other, eventualities are covered by the reference to “promoting” the folding of the polypeptide.

Contacting. The reagents used in the method of the invention require physical contact with the polypeptides whose folding is to be promoted. This contact may occur in free solution, in vitro or in vivo, with one or more components of the reaction immobilised on solid supports. In a preferred aspect, the contact occurs with the molecular chaperone and/or the thiol/disulphide oxidoreductase immobilised on a solid support, for example on a column. Alternatively, the solid support may be in the form of beads or another matrix which may be added to a solution comprising a polypeptide whose folding is to be promoted.

Fragment. When applied to chaperone molecules, a fragment is anything other that the entire native molecular chaperone molecule which nevertheless retains chaperonin activity. Advantageously, a fragment of a chaperonin molecule remains monomeric in solution. Preferred fragments are described below. Advantageously, chaperone fragments are between 50 and 200 amino acids in length, preferably between 100 and 200 amino acids in length and most preferably about 150 amino acids in length.

Unfolded. As used herein, a polypeptide may be unfolded when at least part of it has not yet acquired is correct or desired secondary or tertiary structure. A polypeptide is misfolded when it has acquired an at least partially incorrect or undesired secondary or tertiary structure.

Immobilised, immobilising. Permanently attached, covalently or otherwise. In a preferred aspect of the present invention, the term “immobilise”, and grammatical variations thereof, refer to the attachment of molecular chaperones or, preferably, foldase polypeptides to a solid phase support using a method which comprises a reversible thiol blocking step. This is important where the peptide contains a disulphide. An example of such a method is described herein.

Preferably, before protection the disulphides are reduced using a reducing agent such as DTT (dithiothreitol), under for example an inert gas, such as argon, to prevent reoxidation. Subsequently, the polypeptide is cyanylated, for example using NCTB (2-nitro, 5-thiocyanobenzoic acid) preferably in stoichiometric amounts, and subjected to controlled hydrolysis at high (non-acidic) pH, for example using NaHCO₃. In the case of DsbA, the pH of the hydrolysis reaction is preferably between 6.5 and 10.5 (the pK of DsbA is 4.0), more preferably between 7.5 and 9.5, and most preferably around about 8.5. The thiols are thus reversibly protected.

The polypeptide is then brought into contact with the solid phase component, for example at between 2.0 and 20.0 mg polypeptide/ml of solid component, preferably between 5.0 and 10.0 and most preferably around about 6.5 mg. The coupling is again carried out at a high (non-acidic) pH, for example using an NaHCO₃ coupling buffer. In the case of DsbA, the pH of the coupling reaction is preferably between 6.5 and 10.5, more preferably between 7.5 and 9.5, and most preferably around about 8.5.

Preferably, after coupling the remaining active groups may be blocked, such as with ethanolamine, and the uncoupled polypeptide removed by washing. Thiol groups may finally be regenerated on the coupled polypeptide by removal of the cyano groups, for example by treatment with DTE or DTT.

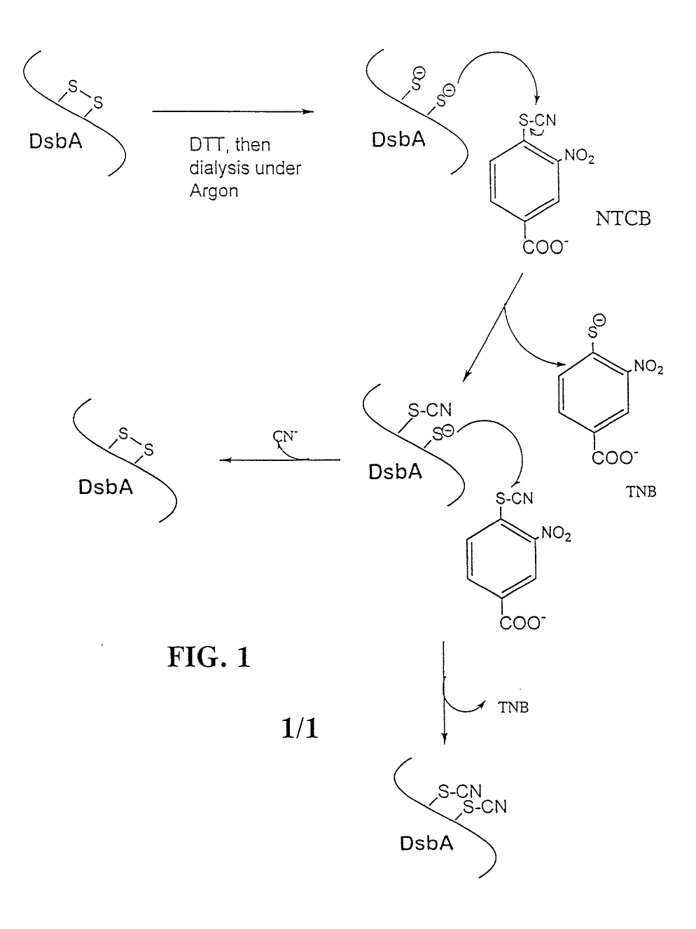

The preferred reaction is shown, schematically, in FIG. 1.

Solid (phase) support. Reagents used in the invention may be immobilised onto solid phase supports. This means that they are permanently attached to an entity which remains in a different (solid) phase from reagents which are in solution. For example, the solid phase could be in the form of beads, a “DNA chip”, a resin, a matrix, a gel, the material forming the walls of a vessel or the like. Matrices, and in particular gels, such as agarose gels, may conveniently be packed into columns. A particular advantage of solid phase immobilisation is that the reagents may be removed from contact with the polypeptide(s) with facility.

Foldase. In general terms, a foldase is an enzyme which participates in the promotion of protein folding through its enzymatic activity to catalyse the rearrangement or isomerisation of bonds in the folding polypeptide. They are thus distinct from a molecular chaperone, which bind to polypeptides in unstable or non-native structural states and promote correct folding without enzymatic catalysis of bond rearrangement. Many classes of foldase are known, and they are common to animals, plants and bacteria. They include peptidyl prolyl isomerases and thiol/disulphide oxidoreductases. The invention comprises the use of all foldases which are capable of promoting protein folding through covalent bond rearrangement.

Moreover, as used herein, the term “a foldase” includes one or more foldases. In general, in the present specification the use of the singular does not preclude the presence of a plurality of the entities referred to, unless the context specifically requires otherwise.

Thiol/disulphide oxidoreductase. As the name implies, thiol/disulphide oxidoreductases catalyse the formation of disulphide bonds and can thus dictate the folding rate of disulphide-containing polypeptides. The invention accordingly comprises the use of any polypeptide possessing such an activity. This includes chaperone polypeptides, or fragments thereof, which may possess PDI activity (Wang & Tsou, (1998) FEBS lett. 425:382-384). In Eukaryotes, thiol/disulphide oxidoreductases are generally referred to as PDIs (protein disulphide isomerases). PDI interacts directly with newly synthesised secretory proteins and is required for the folding of nascent polypeptides in the endoplasmic reticulum (ER) of eukaryotic cells. Enzymes found in the ER with PDI activity include mammalian PDI (Edman et al., 1985, Nature 317:267, yeast PDI (Mizunaga et al. 1990, J. Biochem. 108:848), mammalian ERp59 (Mazzarella et al., 1990, J. Biochem. 265:1094), mammalian prolyl-4-hydroxylase (Pihlajaniemi et al ., 1987, EMBO J. 6: 643) yeast GSBP (Lamantia et al., 1991, Proc. Natl. Acad. Sci. USA, 88:4453) and mammalian T3BP (Yamauchi et al., 1987, Biochem. Biophys. Res. Commun. 146:1485), A. niger PdiA (Ngiam et al., (1997) Curr. genet. 31:133-138) and yeast EUGI (Tachibana et al., 1992, Mol. Cell Biol. 12, 4601). In prokaryotes, equivalent proteins exist, such as the DsbA protein of E. coli. Other peptides with similar activity include, for example, p52 from T. cruzi (Moutiez et al., (1997) Biochem. J. 322:43-48). These polypeptides, and other functionally equivalent polypeptides, are included with the scope of the present invention, as are derivatives of the polypeptides which share the relevant activity (see below). Preferably, the thiol/disulphide oxidoreductase according to the invention is selected from the group consisting of mammalian PDI or E. coli DsbA.

Peptidyl-prolyl isomerase. Peptidyl-prolyl isomerases are known enzymes widely present in a variety of cells. Examples include cyclophilin (see, for example, Bergsma et al. (1991) J. Biol. Chem. 266:23204-23214), parbulen, SurA (Rouviere and Gross, (1996) Genes Dev. 10:3170-3182) and FK506 binding proteins FKBP51 and FKBP52. PPI is responsible for the cis-trans isomerisation of peptidyl-prolyl bonds in polypeptides, thus promoting correct folding. The invention includes any polypeptide having PPI activity. This includes chaperone polypeptides, or fragments thereof, which may possess PPI activity (Wang & Tsou, (1998) FEBS lett. 425:382-384).

Molecular Chaperone. Chaperones, or chaperonins, are polypeptides which promote protein folding by non-enzymatic means, in that they do not catalyse the chemical modification of any structures in folding polypeptides, by promote the correct folding of polypeptides by facilitating correct structural alignment thereof. Molecular chaperones are well known in the art, several families thereof being characterised. The invention is applicable to any molecular chaperone molecule, which term includes, for example, the molecular chaperones selected from the following non-exhaustive group:

p90 Calnexin Salopek et al., J. Investig Dermatol Symp Proc (1996) 1:195

HSP family Walsh et al., Cell Mol. Life Sci. (1997) 53:198

HSP 70 family Rokutan et al., J. Med. Invest. (1998) 44:137

DNA K Rudiger et al., Nat. Struct. Biol. (1997) 4:342

DNAJ Cheetham et al., Cell Stress Chaperones (1998) 3:28

HSP 60 family; GroEL Richardson et al., Trends Biochem. (1998) 23:138

ER-associated chaperones Kim et al., Endoer Rev (1998) 19:173

HSP 90 Smith, Biol. Chem. (1988) 379:283

Hsc 70 Hohfeld, Biol. Chem. (1988) 379:269

sHsps; SecA; SecB Beissinger et al., Biol. Chem. (1988) 379:245

Trigger factor Wang et al., FEBS Lett. (1998) 425:382

zebrafish hsp 47, 70 and Krone et al., Biochem. Cell Biol. (1997) 75:487

HSP 47 Nagata, Matrix Biol. (1998) 16:379

GRP 94 Nicchitta et al., Curr. Opin. Immunol. (1998) 10:103

Cpn 10 Cavanagh, Rev. Reprod. (1996) 1:28

BiP Sommer et al., FASEB J. (1997) 11:1227

GRP 78 Brostrom et al., Prog. Nucl. Acid. res. Mol. Biol. (1998) 58:79

C1p, FtsH Suzuki et al, Trends Biochem. Sci. (1997) 22:118

Ig invariant chain Weenink et al. Immunol. Cell biol. (1997) 75:69

mitochondrial hsp 70 Horst et al., BBA (1997) 1318:71

EBP Hinek, Arch. Immunol. Ther. Exp. (1997) 45:15

mitochondrial m-AAA Langer et al., Experientia (1996) 52:1069

Yeast Ydj1 Lyman et al., Experientia (1996) 52:1042

Hsp 104 Tuite et al., Trends Genet. (1996) 12:467

ApoE Blain et al., Presse Med. (1996) 25:763

Syc Wattiau et al., Mol. Microbiol. (1996) 20:255

Hip Ziegelhoffer et al., Curr. Biol. (1996) 6:272

TriC family Hendrick et al., FASEB J. (1995) 9:1559

CCT Kubota et al., Eur. J. Biochem. (1995) 230:3

PapD, calmodulin Stanfield et aL., Curr. Opin. Struct. Biol. (1995) 5:103

Two major families of protein folding chaperones which have been identified, the heat shock protein 60 (hsp60) class and the heat shock protein 70 (hsp70) class, are especially preferred for use herein. Chaperones of the hsp-60 class are structurally distinct from chaperones of the hsp-70 class. In particular, hsp-60 chaperones appear to form a stable scaffold of two heptamer rings stacked one atop another which interacts with partially folded elements of secondary structure. On the other hand, hsp-70 chaperones are monomers of dimers and appear to interact with short extended regions of a polypeptide.

Hsp70 chaperones are well conserved in sequence and function. Analogues of hsp-70 include the eukaryotic hsp70 homologue originally identified as the IgG heavy chain binding protein (BiP). BiP is located in all eukaryotic cells within the lumen of the endoplasmic reticulum (ER). The prokaryotic DnaK hsp70 protein chaperone in Escherichia coli shares about 50% sequence homology with an hsp70 KAR2 chaperone in yeast (Rose et al. 1989 Cell 57:1211-1221). Moreover, the presence of mouse BiP in yeast can functionally replace a lost yeast KAR2 gene (Normington et al. 19: 1223-1236).

Hsp-60 chaperones are universally conserved (Zeilstra-Ryalls et al., (1991) Ann. Rev. Microbiol. 45:301-325) and include hsp-60 homologues from large number of species, including man. They include, for example, the E. coli GroEL polypeptide; Ehrlichia sennetsu GroEL (Zhang et al., (1997) FEMS Immunol. Med. Microbiol. 18:39-46); Trichomonas vaginalis hsp-60 (Bozner et al., (1997) J. Parasitol. 83:224-229; rat hsp-60 (Venner et al., (1990) NAR 18:5309; and yeast hsp-60 (Johnson et al., (1989) Gene 84:295-302.

In a preferred aspect, the present invention relates to fragments of polypeptides of the hsp-60 family. These proteins being universally conserved, any member of the family may be used; however, in a particularly advantageous embodiment, fragments of GroEL, such as E. coli GroEL, are employed. It has also found that agarose-immobilised calmodulin does have a chaperoning activity, presumably because of its exposed hydrophobic groups.

The sequence of GroEL is available in the art and from academic databases; however, GroEL fragments which conform to the database sequence are inoperative. Specifically, the database contains a sequence in which positions 262 and 267 are occupied by Alanine and Isoleucine respectively. Fragments incorporating one or both of these residues at these positions are inoperative and unable to promote the folding of polypeptides. The invention, instead, relates to a GroEL polypeptide in which at least one of positions 262 and 267 is occupied by Leucine and Methionine respectively.

Derivative. The present invention relates to derivatives of molecular chaperones, peptidyl-prolyl isomerases and thiol/disulphide oxidoreductases. In a preferred aspect, therefore, the terms “molecular chaperone”, “peptidyl-prolyl isomerase” and “thiol-disulphide oxidoreductase” include derivatives thereof which retain the stated activity. The derivatives provided by the present invention include splice variants encoded by mRNA generated by alternative splicing of a primary transcript, amino acid mutants, glycosylation variants and other covalent derivatives of molecular chaperones or foldases which retain the functional properties of molecular chaperones, peptidyl-prolyl isomerases and/or thiol/disulphide oxidoreductases. Exemplary derivatives include molecules which are covalently modified by substitution, chemical, enzymatic, or other appropriate means with a moiety other than a naturally occurring amino acid. Such a moiety may be a detectable moiety such as an enzyme or a radioisotope. Further included are naturally occurring variants of molecular chaperones or foldases found within a particular species, whether mammalian, other vertebrate, yeast, prokaryotic or otherwise. Such a variant may be encoded by a related gene of the same gene family, by an allelic variant of a particular gene, or represent an alternative splicing variant of a molecular chaperone or foldase. Possible derivatives of the polypeptides employed in the invention are described below.

### DESCRIPTION OF PREFERRED EMBODIMENTS

The present invention may be practised in a number of configurations, according to the required use to which the invention is to be put. In a first configuration, the invention relates to the use of a combination of a molecular chaperone and a thiol/disulphide oxidoreductase to facilitate protein folding. The combination of a molecular chaperone and a thiol/disulphide oxidoreductase provides a synergistic effect on protein folding which results in a greater quantity of active, correctly folded protein being produced than would be expected from a merely additive relationship. Advantageously, one or more of the components used to promote protein folding in accordance with the present invention is immobilised on a solid support. However, both molecular chaperones and thiol/disulphide oxidoreductases may be used in solution. They may be used in free solution, but also in suspension, for example bound to a matrix such as beads, for example Sepharose beads, or bound to solid surfaces which are in contact with solutions, such as the inside surfaces of bottles containing solutions, test tubes and the like.

In a second configuration, the invention relates a to the use of a combination of a molecular chaperone and a thiol/disulphide oxidoreductase with a peptidyl prolyl isomerase. The peptidyl prolyl isomerase may be present either bound to a solid support, or in solution. Moreover, it may be bound to beads suspended in solution. The peptidyl prolyl isomerases may be used together with a molecular chaperone alone, with a thiol/disulphide oxidoreductase alone, or with both a molecular chaperone and a thiol/disulphide oxidoreductase. In the latter case, further synergistic effects are apparent over the additive effects which would be expected from the use of the three components together. In particular, an increase in the proportion of the folded protein which is recovered as monodisperse protein, as opposed to aggregated protein, increases substantially.

In a third configuration, the invention relates to the use of an immobilised peptidyl prolyl isomerase for the promotion of protein folding. It has surprisingly been found that peptidyl prolyl isomerase is effective in promoting the folding of unfolded peptides, notwithstanding its previously observed limited effect in accelerating protein folding activity. Immobilised prolyl peptidyl isomerases may be used in combination with molecular chaperones and/or thiol disulphide oxidoreductases, which may be in solution or immobilised as set forth above.

Used in accordance with any of the foregoing configurations, or otherwise in accordance with the following claims, the invention may be used to facilitate protein folding in a variety of situations. For example, the invention may be the used to assist in refolding recombinantly produced polypeptides, which are obtained in an unfolded or misfolded form. Thus, recombinantly produced polypeptides may be passed down a column on which is immobilised a composition comprising protein disulphide isomerase and/or a molecular chaperone and/or a prolyl peptidyl isomerase.

In an alternative embodiment, in a the invention may be employed to maintain the folded conformation of proteins, for example during storage, in order to increase shelf life. under storage conditions, many proteins lose their activity, as a result of disruption of correct folding. The presence of molecular chaperones, in combination with foldases, reduces or reverses the tendency of polypeptides to become unfolded and thus greatly increases the shelf life thereof. In this embodiment, the invention may be applied to reagents which comprise polypeptide components, such as enzymes, tissue culture components, and other proteinaceous reagents stored in solution.

In a third embodiment, the invention may be used to promote the correct folding of proteins which, through storage, exposure to denaturing conditions or otherwise, have become misfolded. Thus, the invention may be used to recondition reagents or other proteins. For example, proteins in need of reconditioning may be passed down a column to which is immobilised a combination of reagents in accordance with he invention. Alternatively, beads having immobilised thereon such a combination may be suspended in a solution comprising the proteins in need of reconditioning. Moreover, the components of the combination according to the invention may be added in solution to the proteins in need of reconditioning.

As noted above, the components of the combination according to the invention may comprise derivatives of molecular chaperones or foldases, including variants of such polypeptides which retain common structural features thereof. Variants which retain common structural features can be fragments of molecular chaperones or foldases. Fragments of molecular chaperones or foldases comprise smaller polypeptides derived from therefrom. Preferably, smaller polypeptides derived from the molecular chaperones or foldases according to the invention define a single feature which is characteristic of the molecular chaperones or foldases. Fragments may in theory be almost any size, as long as they retain the activity of the molecular chaperones or foldases described herein.

With respect to molecular chaperones of the GroEL/hsp-60 family, a preferred set of fragments have been identified which possess the desired activity. These fragments are set forth in our copending international patent application WO99/05163 and in essence comprise any fragment comprising at least amino acid residues 230-271 of intact GroEL, or their equivalent in another hsp-60 chaperone. Preferably, the fragments should not extend beyond residues 150-455 or 151-456 of GroEL or their equivalent in another hsp-60 chaperone. Where the fragments are GroEL fragments, they must not possess the mutant GroEL sequence as set forth above; in other words, they must not have an Alanine residue at position 262 and/or an Isoleucine residue at position 267 of the sequence of intact GroEL.

Advantageously, the fragments comprise the apical domain of GroEL, or its equivalent in other molecular chaperones, or a region homologous thereto as defined herein. The apical domain spans amino acids 191-376 of intact GroEL. This domain is found to be homologous amongst a wide number of species and chaperone types.

This list was compiled from the OWL database release 28.1. The sequences listed below show clear homology to apical domain (residues 191-376) in PDB structure pdb1gr1.ent.

OWL is a non redundant database merging SWISS-PROT, PIR (1-3), GenBank (translation) and NRL-3D.

190-374 CH60_{—}ECOLI 60 KD CHAPERONIN (PROTEIN CPN60) (GROEL PROTEIN)(AMS).—ESCHERICHIA 190-374 CH60_{—}SALTI 60 KD CHAPERONIN (PROTEIN CPN60)(GROEL PROTEIN).—SALMONELLA TYPHI. 191-375 S56371 GroEL protein —Escherichia coli 190-374 CH60_{—}LEPIN 60 KD CHAPERONIN (PROTEIN CPN60) (GROEL PROTEIN) (HEAT SHOCK 58 KD PRO 191-375 S47530 GroEL protein—Porphyromonas gingivalis 190-374 LPNHTPBG NID: g149691—Legionella pneumophila (strain SVir)(library: 189-373 CH60_{—}ACTAC 60 KD CHAPERONIN (PROTEIN CPN60)(GROEL PROTEIN).—ACTINOBACILLUS ACT 191-375 JC4519 heat-shock protein GroEL—Pasteurella multocida

191-375 CH60_{—}BRUAB 60 KD CHAPERONIN (PROTEIN CPN60) (GROEL PROTEIN).—BRUCELLA ABORTUS. 191-375 CH60_{—}HAEIN 60 KD CHAPERONIN (PROTEIN CPN60) (GROEL PROTEIN).—HAEMOPHILUS INFLUE 190-373 CH60_{—}CAUCR 60 KD CHAPERONIN (PROTEIN CPN60)(GROEL PROTEIN).—CAULOBACTER CRESCE 190-374 CH60_{—}AMOPS 60 KD CHAPERONIN (PROTEIN CPN60)(GROEL PROTEIN).—AMOEBA PROTEUS SYM 191-375 CH60_{—}HAEDU 60 KD CHAPERONIN (PROTEIN CPN60)(GROEL PROTEIN).—HAEMOPHILUS DUCREY 191-375 CH61_{—}RHIME 60 KD CHAPERONIN A (PROTEIN CPN60 A)(GROEL PROTEIN A).—RHIZOBIUM ME 190-374 CH60_{—}LEGMI 60 KD CHAPERONIN (PROTEIN CPN60) (GROEL PROTEIN) (58 KD COMMON ANTIGEN 191-375 CH60_{—}YEREN 60 KD CHAPERONIN (PROTEIN CPN60)(GROEL PROTEIN) (HEAT SHOCK PROTEIN 6) 190-374 CH 63_{—}BRAJA 60 KD CHAPERONIN 3 (PROTEIN CPN60 3)(GROEL PROTEIN 3).—BRADYRHIZOBI 191-375 CH60_PORGI 60 KD CHAPERONIN (PROTEIN CPN60) (GROEL PROTEIN).—PORPHYROMONAS GING 191-375 S52901 heat shock protein 60K—Yersinia enterocolitica

191-375 S26423 heat shock protein 60 —Yersinia enterocolitica

191-375 RSU373691 RSU37369 NID: g1208541—Rhodobacter sphaeroides strain=HR. 190-374 CH62_{—}BRAJA 60 KD CHAPERONIN 2(PROTEIN CPN60 2)(GROEL PROTEIN 2).—BRADYRHIZOBI 191-375 CH60_ACYPS 60 KD CHAPERONIN (PROTEIN CPN60)(GROEL PROTEIN)(SYMBIONIN).—ACYRTH 191-375 CH63_{—}RHIME 60 KD CHAPERONIN C(PROTEIN CPN60 C) (GROEL PROTEIN C).—RHIZOBIUM ME 191-375 YEPHSPCRP1 YEPHSPCRP NID: g466575—Yersinia enterocolitica DNA. 191-375 CH60_{—}BORPE 60 KD CHAPERONIN (PROTEIN CPN60) (GROEL PROTEIN).—BORDETELLA PERTUSS 189-373 BRUGRO1 BRUGRO NID: g144106—Brucella aabortus (library: lambda-2001) DNA.

191-375 CH60_{—}PSEAE 60 KD CHAPERONIN (PROTEIN CPN60) (GROEL PROTEIN).—PSEUDOMONAS AERUGI 190-374 CH60_{—}BARBA 60 KD CHAPERONIN (PROTEIN CPN60)(IMMUNOREACTIVE PROTEIN BB65)(IMMUNO 191-375 BAOBB63A NID: g143845—Bartonella bacilliformis (library: ATCC 35685) 189-373 CH60_{—}BACST 60 KD CHAPERONIN (PROTEIN CPN60)(GROEL PROTEIN).—BACILLUS STEAROTHE 188-372

190-373 CH60_{—}BORBU 60 KD CHAPERONIN (PROTEIN CPN60) (GROEL PROTEIN).—BORRELIA BURGDORFE 224-408 S26583 chaperonin hsp60—maize 190-373 A49209 heat shock protein HSP60—Lyme disease spirochete 224-408 MZECPN60B NID: g309558—Zea mays (strain B73) (library:Dashll of P.S 189-373 CH60_THEP3 60 KD CHAPERONIN (PROTEIN CPN60) (GROEL PROTEIN) (HEAT SHOCK 61 KD PRO 188-372 CH60_STAEP 60 KD CHAPERONIN (PROTEIN CPN60)(GROEL PROTEIN) (HEAT SHOCK PROTEIN 6 189-373 CH60_{—}LACLA 60 KD CHAPERONIN (PROTEIN CPN60)(GROEL PROTEIN).—LACTOCOCCUS LACTIS 188-374 CH61_{—}STRAL 60 KD CHAPERONIN 1 (PROTEIN CPN60 1) (GROEL PROTEIN 1) (HSP58).—STRE 191-375 CH60_{—}CHLPN 60 KD CHAPERONIN (PROTEIN CPN60) (GROEL PROTEIN).—CHLAMYDIA PNEUMONI 224-408 MZECPN60A NID: g309556—Zea mays (strain B73) (library:Dach 11 of P. 190-373 HECHSPAB1 HECHSPAB NID: g712829—Helicobacter pylori (individual_isolate 85P) D 221-405 CH60_{—}ARATH MITOCHONDRIAL CHAPERONIN HSP60 PRECURSOR.—ARABIDOPSIS THALIANA (MOUS 224-408 CH60_{—}MAIZE MITOCHONDRIAL CHAPERONIN HSP60 PRECURSOR.—ZEA MAYS (MAIZE). 190-374 CH60_{—}CHLTR 60 KD CHAPERONIN (PROTEIN CPN60)(GROEL PROTEIN)(57 KD CHLAMYDIAL HYP 189-373 CH60_{—}STAAU 60 KD CHAPERONIN (PROTEIN CPN60) (GROEL PROTEIN) (HEAT SHOCK PROTEIN 6 189-373 CH60_{—}CLOPE 60 KD CHAPERONIN (PROTEIN CPN60)(GROEL PROTEIN).—CLOSTRIDIUM PERFRI 212-397 HS60_YEAST HEAT SHOCK PROTEIN 60 PRECURSOR (STIMULATOR FACTOR 1 66 KD COMPONENT) 217-403 CH60_{—}PYRSA 60 KD CHAPERONIN (PROTEIN CPN60)(GROEL PROTEIN).—PYRENOMONAS SALINA 191-377 CH60_{—}EHRCH 60 KD CHAPERONIN (PROTEIN CPN60) (GROEL PROTEIN).—EHRLICHIA CHAFFEEN 191-375 CHTGROE1 CHTGROE NID: g144503—C.trachomatis DNA. 188-372 CH60_{—}THETH 60 KD CHAPERONIN(PROTEIN CPN60) (GROEL PROTEIN).—THERMUS AQUATICUS 189-373 TAU294831 TAU29483 NID: g1122940—Thermus aquaticus. 190-378 CH60_{—}RICTS 60 KD CHAPERONIN (PROTEIN CPN60) (GROEL PROTEIN) (MAJOR ANTIGEN 58) (5 189-375 SYCCPNC SYCCPNC NID: g1001102—Synechocystis sp. (strain PCC6803,) DNA.

190-373 CPU308211 CPU30821 NID: g1016083—Cyanophora paradoxa. 189-373 CH61_{—}MYCLE 60 KD CHAPERONIN 1 (PROTEIN CPN60 1) (GROEL PROTEIN 1).—MYCOBACTERIU 239-423 PSU21139 PSU21139 NID: g806807—pea. 191-377 CH60_{—}COWRU 60 KD CHAPERONIN (PROTEIN CPN60)(GROEL PROTEIN).—COWDRIA RUMINANTIU 245-429 RUBB_{—}BRANA RUBISCO SUBUNIT BINDING-PROTEIN BETA SUBUNIT PRECURSOR (60 KD CHAPERON 144-328 SCCPN60 SCCPN60 NID: g1167857—rye.

153-338 CH60_{—}EHRRI 60 KD CHAPERONIN (PROTEIN CPN60) (GROEL PROTEIN) (55 KD MAJOR ANTIGEN) 245-429 RUBB_ARATH RUBISCO SUBUNIT BINDING-PROTEIN BETA SUBUNIT PRECURSOR (60 KD CHAPERON 235-419 ATU49357 ATU49357 NID: g1223909—thale cress strain=ecotype Wassilewskija. 195-379 RUB1_BRANA RUBISCO SUBUNIT BINDING-PROTEIN ALPHA SUBUNIT (60 KD CHAPERONIN ALPHA 189-374 CH62_SYNY3 60 KD CHAPERONIN 2 (PROTEIN CPN60 2) (GROEL HOMOLOG 2).—SYNECHOCYSTI 178-362 RUBA_RICCO RUBISCO SUBUNIT BINDING-PROTEIN ALPHA SUBUNIT (60 KD CHAPERONIN ALPHA 190-375 CH60_{—}ODOSI 60 KD CHAPERONIN (PROTEIN CPN60) (GROEL PROTEIN).—ODONTELLA SINENSIS 236-420 PSU21105 PSU21105 NID: g1185389—pea. 224-409 CH60_BRANA MITOCHONDRIAL CHAPERONIN CH60_{—}BACSU 60 KD CHAPERONIN (PROTEIN CPN60)(GROEL PROTEIN).—BACILLUS SUBTILIS. 191-375 CH60_{—}AGRTU 60 KD CHAPERONIN (PROTEIN CPN60)(GROEL PROTEIN).—AGROBACTERIUM TUME 191-375 b36917 heat shock protein GroEL—Agrobacterium tumefaciens

191-375 PAU17072 PAU17072 NID: g576778—Pseudomonas aeruginosa. 191-375 CH60_{—}RHILV 60 KD CHAPERONIN (PROTEIN CPN60)(GROEL PROTEIN).—RHIZOBIUM LEGUMINO 187-373 CH61_{—}STRCO 60 KD CHAPERONIN 1 (PROTEIN CPN60 1)(GROEL PROTEIN 1)(HSP58).—STRE 191-375 CH60_{—}COXBU 60 KD CHAPERONIN (PROTEIN CPN60) (GROEL PROTEIN) (HEAT SHOCK PROTEIN B 191-375 CH62_{—}RHIME 60 KD CHAPERONIN B (PROTEIN CPN60 B) (GROEL PROTEIN B).—RHIZOBIUM ME 191-375 PSEGROESL1 PSEGROESL NID: g151241—Pseudomonas aeruginosa (library: ATCC 27853) 189-372 CH61_SYNY3 60 KD CHAPERONIN 1 (PROTEIN CPN60 1)(GROEL HOMOLOG 1).—SYNECHOCYSTI 189-373 CH60_{—}CLOTM 60 KD CHAPERONIN (PROTEIN CPN60)(GROEL PROTEIN)(HSP-60).—CLOSTRIDI 191-373 CH60_{—}PSEPU 60 KD CHAPERONIN (PROTEIN CPN60)(GROEL PROTEIN).—PSEUDOMONAS PUTIDA 190-373 CH60_SYNP7 60 KD CHAPERONIN (PROTEIN CPN60) (GROEL PROTEIN).—SYNECHOCOCCUS SP. 190-374 CH60_{—}GALSU 60 KD CHAPERONIN (PROTEIN CPN60) (GROEL PROTEIN).—GALDIERIA SULPHURA 190-374 CH60_{—}ZYMMO 60 KD CHAPERONIN (PROTEIN CPN60) (GROEL PROTEIN).—ZYMOMONAS MOBILIS. 191-375 JC2564 heat shock protein groEL—Zymomonas mobilis

191-375 CH60_{—}CHRVI 60 KD CHAPERONIN (PROTEIN CPN60) (GROEL PROTEIN).—CHROMATIUM VINOSUM 189-373 CH60_{—}MYCTU 60 KD CHAPERONIN (PROTEIN CPN60)(GROEL PROTEIN)(65 KD ANTIGEN)(HEAT 191-375 CH60_{—}NEIME 60 KD CHAPERONIN (PROTEIN CPN60) (GROEL PROTEIN)(63 KD STRESS PROTEIN 189-373 CH60_{—}TREPA 60 KD CHAPERONIN (PROTEIN CPN60)(GROEL PROTEIN)(TPN60)(TP4 ANTIGEN) 190-374 CH60_{—}HELPY 60 KD CHAPERONIN (PROTEIN CPN60) (GROEL PROTEIN) (HEAT SHOCK PROTEIN 6 191-375 CH60_{—}NEIGO 60 KD CHAPERONIN (PROTEIN CPN60) (GROEL PROTEIN) (63 KD STRESS PROTEIN 222-406 CH61_{—}CUCMA MITOCHONDRIAL CHAPERONIN HSP60-1 PRECURSOR.—CUCURBITA MAXIMA (PUMPKI 189-373 CH60_MYCPA 60 KD CHAPERONIN (PROTEIN CPN60)(GROEL PROTEIN)(65 KD ANTIGEN)(HEAT 230-414 MPU15989 MPU15989 NID:g559802—Mycobacterium paratuberculosis. 224-408 S26582 chaperonin hsp60—maize 191-375 S40247 heat-shock protein—Neisseria gonorrhoeae 189-373 CH60_{—}CLOAB 60 KD CHAPERONIN (PROTEIN CPN60)(GROEL PROTEIN).—CLOSTRIDIUM ACETOB 191-375 CH60_{—}NEIFL 60 KD CHAPERONIN (PROTEIN CPN60)(GROEL PROTEIN)(63 KD STRESS PROTEIN 190-373 CH60_{—}LEGPN 60 KD CHAPERONIN (PROTEIN CPN60) (GROEL PROTEIN) (58 KD COMMON ANTIGEN 222-406 CH62_{—}CUCMA MITOCHONDRIAL CHAPERONIN HSP60-2 PRECURSOR.—CUCURBITA MAXIMA (PUMPKI 191-375 CHTGROESL1 CHTGROESL NID: g402332—Chlamydia trachomatis DNA. 64-248 S40172 S40172 NID: g251679—Chlamydia psittaci pigeon strain P-1041. 189-373 SYOGROEL2 SYOGROEL2 NID:g562270—Synechococcus vulcanus DNA. 191-375 CH60_{—}CHLPS 60 KD CHAPERONIN (PROTEIN CPN60)(GROEL PROTEIN)(57 KD CHLAMYDIAL HYP 188-372 CH62_{—}STRAL 60 KD CHAPERONIN 2 (PROTEIN CPN60 2)(GROEL PROTEIN 2)(HSP56). —STRE 189-373 CH62_{—}MYCLE 60 KD CHAPERONIN 2 (PROTEIN CPN60 2) (GROEL PROTEIN 2) (65 KD ANTIGEN) 236-420 MSGANTM MSGANTM NID: g149923—M.leprae DNA, clone Y3178.

CPN60 PRECURSOR.—BRASSICA NAPUS (RAPE). 105-289 PMSARG2 PMSARG2 NID: g607157—Prochlorococcus marinus.

234-417 RUB2_BRANA RUBISCO SUBUNIT BINDING-PROTEIN ALPHA SUBUNIT PRECURSOR (60 KD CHAPERO 75-259 CRECPN1A CRECPN1A NID: g603910—Chlamydomonas reinhardtii cDNA to mRNA. 215-400 P60_CRIGR MITOCHONIDRIAL MATRIX PROTEIN P1 PRECURSOR (P60 LYMPHOCYTE PROTEIN) (CH224-408 CRECPN1B CRECPN1B NID: g603912—Chlamydomonas reinhardtii cDNA to mRNA. 191-375 RUBA_WHEAT RUBISCO SUBUNIT BINDING-PROTEIN ALPHA SUBUNIT PRECURSOR (60 KD CHAPERO 189-373 B47292 heat shock protein groEL—Mycobacterium tuberculosis

206-391 CELHSP60CP CELHSP60CP NID: g533166—Caenorhabditis elegans (strain CB1392) cDNA 215-400 P60_HUMAN MITOCHONDRIAL MATRIX PROTEIN P1 PRECURSOR (P60 LYMPHOCYTE PROTEIN)(CH 215-400 P60_MOUSE MITOCHONDRIAL MATRIX PROTEIN P1 PRECURSOR (P60 LYMPHOCYTE PROTEIN) (CH 215-400 P60_RAT MITOCHONDRIAL MATRIX PROTEIN P1 PRECURSOR (P60 LYMPHOCYTE PROTEIN) (CH 215-400 A41931 chaperonin hsp6—mouse

197-382 MMHSP60A MMHSP60A NID:g51451—house mouse. 218-402 CH63_HELVI 63 KD CHAPERONIN PRECURSOR (P63).—HELIOTHIS VIRESCENS (NOCTUID MOTH) 205-390 EGHSP60GN EGHSP60GN NID: g1217625—Euglena gracilis. 222-407 HS60_SCHPO PROBABLE HEAT SHOCK PROTEIN 60 PRECURSOR.—SCHIZOSACCHAROMYCES POMBE 198-385 S61295 heat shock protein 60—Trypanosoma cruzi 198-385 TRBMTHSP TRBMTHSP NID: g903883—Mitochondrion Trypanosoma brucei (strain EATRO 8-69 ECOGROELA ECOGROELA NID: g146268—E.coli DNA, clone E. 142-325 ENHCPN60P ENHCPN60P NID: g675513—Entamoeba histolytica (strain HM-1:IMSS) DNA. 257-433 CH60_PLAFG MITOCHONDRIAL CHAPERONIN CPN60 PRECURSOR.—PLASMODIUM FALCIPARUM (ISO 1-90 CRECPN1C CRECPN1C NID: g603914—Chlamydomonas reinhardtii cDNA to mRNA.

5-65 ATTS0779 ATTS0779 NID: gl7503—thale cress. 189-373 CH60_{—}MYCGE 60 KD CHAPERONIN (PROTEIN CPN60) (GROEL PROTEIN).—MYCOPLASMA GENITAL 228-411 HTOHSP60X HTOHSP60X NID: g553068—Histoplasma capsulatum (strain G217B) DNA.

190-297 CH60_SYNP6 60 KD CHAPERONIN (PROTEIN CPN60) (GROEL PROTEIN)(FRAGMENT).—SYNECHO 169-245 RUBA_ARATH RUBISCO SUBUNIT BINDING-PROTEIN ALPHA SUBUNIT (60 KD CHAPERONIN ALPHA.

Such analyses may be repeated using other databases, or more recent updates of the OWL database, and for other chaperone families, such as the HSP 70, HSP 90 or GRP families.

Preferably, molecular chaperones according to the invention are homologous to, or are capable of hybridising under stringent conditions with, a region corresponding to the apical domain of GroEL as defined above. p In a highly preferred embodiment, the fragments are selected from the group consisting of residues 191-376, 191-345 and 191-335 of the sequence of intact GroEL.

Derivatives of the molecular chaperones or foldases also comprise mutants thereof, including mutants of fragments and other derivatives, which may contain amino acid deletions, additions or substitutions, subject to the requirement to maintain the activity of the molecular chaperones or foldases described herein. Thus, conservative amino acid substitutions may be made substantially without altering the nature of the molecular chaperones or foldases, as may truncations from the 5′ or 3′ ends. Deletions and substitutions may moreover be made to the fragments of the molecular chaperones or foldases comprised by the invention. Mutants may be produced from a DNA encoding a molecular chaperone or foldase which has been subjected to in vitro mutagenesis resulting e.g. in an addition, exchange and/or deletion of one or more amino acids. For example, substitutional, deletional or insertional variants of molecular chaperones or foldases can be prepared by recombinant methods and screened for immuno-crossreactivity with the native forms of the relevant molecular chaperone or foldase.

The fragments, mutants and other derivative of the molecular chaperones or foldases preferably retain substantial homology with the native molecular chaperones or foldases. As used herein, “homology” means that the two entities share sufficient characteristics for the skilled person to determine that they are similar in origin and function. Preferably, homology is used to refer to sequence identity. Thus, the derivatives of molecular chaperones or foldases preferably retain substantial sequence identity with native forms of the relevant molecular chaperone or foldase.

“Substantial homology”, where homology indicates sequence identity, means more than 40% sequence identity, preferably more than 45% sequence identity and most preferably a sequence identity of 50% or more, as judged by direct sequence alignment and comparison.

Sequence homology (or identity) may moreover be determined using any suitable homology algorithm, using for example default parameters. Advantageously, the BLAST algorithm is employed, with parameters set to default values. The BLAST algorithm is described in detail at http://www.ncbi.nih.gov/BLAST/blast_help.html, which is incorporated herein by reference. The search parameters are defined as follows, and are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (see http://www.ncbi.nih.gov/BLAST/blast_help.html) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al. (1994) Nature Genetics 6:119-129.

The five BLAST programs available at http://www.ncbi.nlm.nih.gov perform the following tasks:

blastp compares an amino acid query sequence against a protein sequence database;

blastn compares a nucleotide query sequence against a nucleotide sequence database;

blastx compares the six-frame conceptual translation products of a nucleotide query sequence (both strands) against a protein sequence database;

tblastn compares a protein query sequence against a nucleotide sequence database dynamically translated in all six reading frames (both strands).

tblastx compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual).

DESCRIPTIONS Restricts the number of short descriptions of matching sequences reported to the number specified; default limit is 100 descriptions. (See parameter V in the manual page). See also EXPECT and CUTOFF.

ALIGNMENTS Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Most preferably, sequence comparisons are conducted using the simple BLAST search algorithm provided at http://www.ncbi.nlm.nih.gov/BLAST.

Alternatively, sequence similarity may be defined according to the ability to hybridise to a complementary strand of a chaperone or foldase sequence as set forth above.

Preferably, the sequences are able to hybridise with high stringency. Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1 M Na+ at 65-68 ° C. High stringency conditions can be provided, for example, by hybridisation in an aqueous solution containing 6×SSC, 5×Denhardt's, 1 % SDS (sodium dodecyl sulphate), 0.1 Na+ pyrophosphate and 0.1 mg/ml denatured salmon sperm DNA as non specific competitor. Following hybridisation, high stringency washing may be done in several steps, with a final wash (about 30 min) at the hybridisation temperature in 0.2 -0.1×SSC, 0.1 % SDS.

Moderate stringency refers to conditions equivalent to hybridisation in the above described solution but at about 60-62° C. In that case the final wash is performed at the hybridisation temperature in 1×SSC, 0.1 % SDS.

Low stringency refers to conditions equivalent to hybridisation in the above described solution at about 50-52° C. In that case, the final wash is performed at the hybridisation temperature in 2×SSC, 0.1 % SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the probe also play a role.

The invention also envisages the administration of combinations according to the invention as compositions, preferably for the treatment of diseases associated with protein misfolding. The active compound may be administered in a convenient manner such as by the oral, intravenous (where water soluble), intramuscular, subcutaneous, intranasal, intradermal or suppository routes or implanting (e.g. using slow release molecules). Depending on the route of administration, the active ingredient may be required to be coated in a material to protect said ingredients from the action of enzymes, acids and other natural conditions which may inactivate said ingredient.

In order to administer the combination by other than parenteral administration, it will be coated by, or administered with, a material to prevent its inactivation. For example, the combination may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin.

Liposomes include water-in-oil-in-water CGF emulsions as well as conventional liposomes.

The active compound may also be administered parenterally or intraperitoneally.

Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thirnerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compound in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the combination of polypeptides is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. The amount of active compound in such therapeutically useful compositions in such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In a further aspect there is provided the combination of the invention as hereinbefore defined for use in the treatment of disease. Consequently there is provided the use of a combination of the invention for the manufacture of a medicament for the treatment of disease associated with aberrant protein/polypeptide structure. The aberrant nature of the protein/polypeptide may be due to misfolding or unfolding which in turn may be due to an anomalous e.g. mutated amino acid sequence. The protein/polypeptide may be destabilised or deposited as plaques e.g. as in Alzheimer's disease. The disease might be caused by a prion. A polypeptide-based medicament of the invention would act to renature or resolubilise aberrant, defective or deposited proteins.

The results presented herein show that using the refolding matrix and refolding method of the present invention, it is possible to obtain a refolded polypeptide having at least 100% of the activity of the corresponding native protein, in some case greater than 100% of the activity of the corresponding native protein. Thus, in another aspect, the present invention provides a polypeptide obtained by the method of the invention. Preferably said polypeptide has at least 100% of the activity of the corresponding native protein, more preferably greater than 100%.

Preferably, the polypeptide has been obtained by using the ternary matrix of the present invention.

For the purposes of comparison, activity, such as biological activity, is typically determined for equal amounts of the native protein, optionally purified, and the refolded protein. By equal amounts, it is generally meant equal amounts of soluble protein in terms of mass.

In a preferred embodiment, the polypeptide is a toxin polypeptide, more preferably a scorpion toxin polypeptide, such as Cn5 toxin.

Polypeptides obtained by the method of the invention may be formulated for use in therapeutic applications as described above for the molecular chaperone/foldase combinations. Thus a refolded polypeptide obtained by the method of the invention may be formulated as a pharmaceutical composition. Therapeutic uses for refolded proteins will generally be similar to those of the corresponding native or recombinant (but less active) protein.

The invention is further described below, for the purposes of illustration only, in the following Examples:

### EXAMPLE 1

Mixed Bed Mini-Chaperone/DsbA/Cyclophilin Gels

Expression, purification and immobilisation of the mini-chaperone. The mini-chaperone (191-345 peptide fragment from E. coli GroEL), is cloned and expressed in E. coli as a fusion protein containing a 17-residue N-terminal histidine tail (Zahn et al. (1996) Proc. Natl. Acad. Sci. USA 93, 15024-15029). The mini-chaperone is immobilised on agarose gel beads as previously reported (Altamirano et al. (1997) Proc. Natl. Acad. Sci, USA. 94, 3576-3578) except that NHS-activated Sepharose-4 Fast Flow (Pharmacia Biotech, Sweden) is used. This activated gel, which has a longer spacer arm than that used in our former preparation, is more efficient and stable. Leakage is reduced to zero and the capacity to refold cyclophilin A, is increased to 6 mg of substrate per mL of wet gel, that is 1.5 times the value for the previously reported refolding gel.

Expression, Purification and Immobilisation of Human PPI

Human PPI (peptidyl-prolyl cis-trans-isomerase) is expressed and purified as described (Jasanoff et al. (1994) Biochemistry 33, 6350-6355) with some minor modifications. Briefly a plasmid carrying the gene of fusion protein GST-PPI is used to transform the E. coli C41 D3 strain (Miroux and Walker (1996) J. Mol. Biol 260, 289-298). The cells are grown in 2×TY medium at 34° C. Innoculae are grown up to A₆₀₀=0.5 before induction with 0.7 mM isopropyl β-D-thiogalactoside and the cultures are allowed to grow for 16 h at 25° C. before being harvested. The cell pellet is resuspended in buffer (50 mM sodium phosphate, pH 7.5, 100 mM NaCl, 1% Triton X100 and 0.2 mM PMSF), sonicated to release proteins, and the protein is purified by affinity chromatography using glutathione agarose. The bound fusion protein is then treated with thrombin on the column to obtain free PPI. The thrombin also present in the eluate is removed by affinity chromatography on benzamidine agarose. The purity of the PPI is verified by SDS-PAGE and FPLC using a Superdex 75 column (Pharmacia Biotech). PPI is assayed as previously described and bound to NHS-Sepharose 4 fast flow as described above for mini-chaperone immobilisation.

Cloning, Expression, and Purification of DsbA

The E. coil dsbA gene is amplified by PCR using dsbA-Fo and dsbA-Ba primers, based on its known sequence. The amplified whole expressed gene, including its signal peptide is digested with NcoI and BamH1 and cloned into the high expression plasmid pCE820 (Lewis et al. (1993) Bioorganic & Medicinal Chemistry Letters. 3, 1197-1202). The pMA14 (pCE820-DsbA) is purified and the sequence is confirmed by standard sequencing techniques. The dsbA gene product is overproduced in the E. coli C41 D3 strain (Miroux and Walker, 1996) and appears almost exclusively in the periplasmic fraction. The cells are grown in 2×TY medium at 37° C. Innoculae are grown up to A₆₀₀=0.2 before induction with 0.7 mM isopropyl β-D-thiogalactoside and the cultures are allowed to grow for 12-14 h at 30° C. before being harvested. Cell proteins are fractionated in spheroplasts and the resulting soluble periplasm contents is prepared by using the lysozyme/EDTA method. The suspension containing the spheroplasts is centrifuged (48,000×g, 30 min, at 4° C.). Proteins are desalted in 10 mM MOPS/NaOH, pH 7.0 by diafiltration using 10 kDa cut-off membranes in a tangential flow system (Minisette, Filtron). DsbA protein is purified by ion-exchange chromatography using a Mono-Q HR 10/10 FPLC column (Pharmacia, Biotech) which is eluted with a shallow KCl gradient (0-250 mM). DsbA emerges at about 70 mM KCl an is>95% pure as shown by SDS-PAGE (20% gels) and also by gel filtration chromatography (Superdex 75, Pharmacia Biotech). The concentration of DsbA protein is calculated from its absorption at 280 nm, using the absorption coefficient A₂₈₀, 1 mg/mL/cm=1.10 for the native oxidised protein. The activity of the soluble DsbA protein is determined by using the spectrofluorometric method described by Wunderlich (1993).

Reversible Blocking of Cys-30 in DsbA Protein in an Inert Atmosphere

All the experiments are performed in a glove box in an argon (Ar) atmosphere and the solution reagents are pre-saturated with Ar. The disulphide group at the active site of DsbA is reduced with 5 mM DTT, in 25 mM MES-K⁺ buffer pH 6.0 for 1 h; DTT is then removed by dialysis under Ar to avoid reoxidation. DsbA is then cyanylated under Ar with NTCB (2-nitro-5-thiocyanate benzoate) (Altamirano, et al. (1989) Arch. Biochim. Biophys. 269, 555-561; Altamirano et al. (1992) Biochemistry 31, 1153-1158) at a final concentration of 5 mM. The reaction is practically instantaneous and it is apparent from the appearance of a yellow colour from the departing group, the anion 2-nitro-5-thiobenzoate. After 30 min the extent of the reaction is evaluated by measuring its absorption at 412 um (e₄₁₂=14,140 M⁻¹ cm⁻¹) and it is found to be stoichiometric (Altamirano et al, 1992). The protein is chromatographically desalted (desalt 10/10 column, Pharmacia Biotech) in 50 mM NaHCO₃ buffer, pH 8.3/0.5 M KCl.

Attachment to NHS-Activated Sepharose-4 Fast Flow Gel

5 mL of wet gel (NHS-activated sepharose-4 fast flow from Pharmacia Biotech, Sweden) is washed with 15 volumes of cold 1 mM HCl and then suspended in 50 mM NaHCO₃ at pH 8.3/0.5 M KCl, mixed in an end-over-end shaker for 1 min at room temperature. DsbA protein, with its thiols reversibly blocked, is added to the gel suspension (7 mg protein/mL gel) and mixed in an end-over-end shaker for 2 h at room temperature. It is then washed with the coupling buffer. The remaining active groups are blocked by adding 2.5 M ethanolamine at pH 8 and mixing at room temperature for 4 h. Uncoupled DsbA is removed by washing with five cycles of alternately high and low pH buffer solution (Tris-HCl 0.1M pH 7.8 containing 0.5 M NaCl followed by acetate buffer, 0.1M, pH 4 plus 0.5 M NaCl). The gel is finally washed with 5-10 gel volumes of refolding buffer (see below) and SH groups regenerated by treatment with DTT. The gel is washed with ten times gel volume of refolding buffer. After this, the immobilised DsbA protein is oxidised as detailed under experimental protocol. The coupling efficiency of this procedure is higher than 95 %.

All the refolding experiments are performed in a batch mode. After use, the gel is regenerated by washing with 5 volumes of stored buffer (100 mM sodium phosphate pH 8+2 mM EDTA +0.5 M KCl). The gel is stable for at least one year when stored at 4° C. in 100 mM sodium phosphate pH 7.0, containing 2 mM EDTA.

Mixed Bed Mini-Chaperone/DsbA Gels

Two approaches are used to prepare a combined matrix of mini-chaperone, PPI and/or PDI:

a) each protein is separately immobilised on NHS-Sepharose and the gels are thoroughly mixed; or

b) the proteins are mixed, and immobilised on NHS-agarose.

Comparable results are initially obtained with both kinds of refolding gel. Most of the following data are obtained from experiments using gels of type b).

For testing these gels for refolding chromatography of proteins containing disulphide bridges that are very difficult to refold in vitro two examples are selected: the scorpion toxin CN5 and a single chain antibody, which have previously been particularly difficult to fold.

### EXAMPLE 2

Refolding of Scorpion Toxin on a Minichaperone/PDI Gel

The crustacean-specific toxin Cn5, isolated from the venom of the scorpion Centruroides noxius is used. This peptide contains 66 amino acid residues and is stabilised by four disulphide bridges: Cys12-Cys65, Cys16-Cys41, Cys25-Cys46 and Cys29-Cys48. Toxicity tests have previously revealed that Cn5 is a toxin that affects arthropods but not mammals.

Refolding Conditions

A sample of the pure denatured toxin is obtained from the laboratory of Dr. L. Possani, Institute of Biotechnology, Cuernavaca, Mor., Mexico. The refolding protocol is as follows:

1. The lyophilised protein is dissolved in 8M urea+0.3 M DTE and dialysed against 6M GnHCl (pH 2.0) at 23° C. for 2 h in order to maintain the thiols in their reduced state.

2. 3.5 nmol of denatured Cn5 (25 μg) are diluted 200 times in a gel slurry previously equilibrated with the “refolding buffer” (100 mM potassium phosphate buffer ()H 7.7) 0.5M L-arginine, 1 mM GSH (=glutathione), 1 mM GSSG, 2 mM EDTA). The mixture is gently mixed by upside down rotation, and kept under rotation for 5 h at room temperature.

The gel is packed into a small column and eluted with refolding buffer. Then it is concentrated by ultrafiltration under pressure (Amicon cell) changing the buffer to 5 mM phosphate pH 7.7 (final concentration 5 mM).

The preparation is eventually lyophilised.

Simultaneously, the following controls and experiments are performed:

a) Cn5 diluted 1:200 in refolding buffer alone.

b) The same as a), plus mini-chaperone-agarose (fragment 191-345),

c) The same as a), plus DsbA-agarose

d) The same as a), plus combined gel containing DsbA and fragment 191-345.

Only the samples treated as d) yielded soluble protein. This is tested for toxicity and is found to be as toxic as the native peptide for the crustacean Procambarus bouvieri.

### EXAMPLE 3

Refolding of a Single-Chain Recombinant Antibody (ScFv) on a Minichaperone/PDI Gel

The ScFv (31 kDa) with two disulphide bridges is a recombinant antibody that is derived from a mouse monoclonal hybridoma line with anti-rhodopsin specificity (against the C-terminus of rhodopsin).

The denatured protein, obtained from Dr. C. Smith Laboratory (University of Florida, Gainesville, Fla., USA.) had been partially purified from inclusion bodies, and is received in 6M GnHCl+0.5 M imidazole buffer. The buffer is changed to 6M GnHCl and 25 mM ammonium acetate, pH 5.0, 0.3 M DTE added and left standing for 2 h. The sample is diluted in the following refolding buffer (100 mM Tris-HCI, 0.5M L-arginine, 2 mM EDTA, 8 mM GSSG) and divided in six samples:

A=control Oust refolding buffer)

B=Segment 191-376-agarose

C=Segment 191-345-agarose

D Segment 191-376-agarose+DsbA-agarose

E=Segment 191-345-agarose+DsbA-agarose

F DsbA-agarose

Batchwise Renaturation of ScFv

A solution of denatured ScFv in 6 M GnHCl+0.3 M DTT is diluted 100-fold in the refolding buffer under conditions A-F (above) After gently mixing for 12 h, t a column is packed and eluted with the refolding buffer plus 150 mM NaCl. After refolding the samples are dialysed against 50 mM phosphate pH 7.7+150 mM NaCl and tested by western blot and ELISA. ScFv obtained according to E is by far the most active in both assays, showing specificity for rhodopsin in the ELISA test.

### EXAMPLE 4

Refolding of Cn5 Toxin in Binary (Minichaperone/PDI) and Ternary (Minichaperone/PDI/PPI) Gels

Activity of Immobilised DsbA

In all these analyses, the activity of soluble DsbA protein is measured as a control. Two methods are used.

Reduction of Insulin. Catalysis of the reduction of insulin by DTT is assayed according to Holmgren (1979), J. Biol. Chem. 254, 9627-9632. For immobilised DsbA protein, 50 mL of beads containing the DsbA protein are added (1.2 nmol) into 2.0 mL reaction mixture. After 10 min of gentle mixing, the resin is left to sediment by gravity before measuring the turbidity of the supernatant at 650 nm. Measurements of the scattered light at 350 mn are performed using a Hitachi 4000 spectrofluorimeter.

Assay of Disulphide Exchange of Scrambled RNAseA

Reduced RNAse (rRNAse) and scrambled oxidised RNAse (sRNAse) are obtained and refolding assays are performed, according to Lyles and Gilbert, (1991) Biochemistry 30, 613-619.

Cn5 Toxin Purification. Soluble venom from the scorpion Centruroides noxius Hoffmann is purified by three sequential chromatographic steps as described (Garcia et al. (1997) Comparative Biochemistry and physiology B-Biochemistry & Molecular Biology 116, 315-322).

Batchwise Renaturation of Cn5 Scorpion Toxin

Denatured and Reduced Cn5

The lyophilised Cn5 toxin (250 mg) is dissolved in 100 mL of 6 M guanidinium chloride prepared in 0.1M potassium phosphate buffer (pH 8). It is then, reduced with 0.1 M DTT and left for 3 h at 23° C. to ensure the completeness of the reaction. The toxin is then dialysed against 6 M guanidinium chloride prepared in 0.1 M potassium phosphate buffer (pH 3), adjusted with phosphoric acid, in order to maintain the thiol groups in their reduced state. The fluorescence and CD spectrum of reduced and denatured Cn5 toxin are the typical ones for a denatured protein. The quantitative reduction of Cn5 is verified by the determination of free sulfhydryls with DTNB (5, 5′-dithiobis(2-nitrobenzoic acid) and 8 Cys residues per chain are found.

Refolding Matrix and Folding of Cn5 Toxin

The binary refolding matrix is a 1:1 mixture of mini-chaperone and DsbA; the ternary refolding matrix is obtained by mixing equal concentrations of mini-chaperone, DsbA protein and PPI. Both kinds of refolding gels are equilibrated with pH 8 buffer prepared with 100 mM potassium phosphate, 0.5 M L-arginine, 1 mM GSSG (glutathione oxidised form), 1 mM GSH (glutathione reduced form) and 2 mM sodium EDTA (refolding buffer). In all cases, the denatured and reduced Cn5 is added very slowly, mixed and diluted 100-fold with a resuspension of the binary or the ternary refolding matrix, and kept under gentle mixing at 20° C. After 4 h, the gel suspension is then centrifuged to separate the supernatant. The gel pellet is washed with refolding buffer containing 0.5 M KCl. The preparations are eventually concentrated, chromatographically desalted for replacing the refolding buffer by water or 50 mM ammonium acetate buffer (pH 5.5) and then lyophilised. For biological assays, the toxin is dissolved in water.

Each of the three refolding proteins (mini-chaperone or DsbA or PPI) used is also individually tested and a control experiment is also made using refolding buffer alone (Table I).

CD Studies of the Refolded Cn5 and its Denatured State

CD spectra are obtained using a Jasco (Easton, MD) Model J-720 spectrometer with a spectral resolution of 0.2 nm. CD calibration is performed using (1S)-(+)-10-camphor-sulfonic acid (Aldrich) with a molar extinction coefficient of 34.5 M⁻¹ cm⁻¹ at 285 nm and a molar ellipticity of 2.36 M⁻¹cm⁻¹ at 290.5 nm. The CD spectrum are recorded using an enzyme concentration of 0.05 mg/mL in 25 mM potassium phosphate buffer, pH 8, in a 0.1 cm stress-free cuvette at room temperature.

Cn5 Bio-assays. Lethality tests are performed on the land crustacean Armadillidium vulgare (pill bug) in the laboratory of Lourival D. Possani, Departamento de Reconocimiento Estructural, Instituto de Biotecnologia, UNAM PO Box 510-3, Cuernavaca, MOR, 62250, México.

LD₅₀ Determination for Native Cn5

Five groups of 6 animals each are used. The control group is injected with 5 μL of water. Different amounts of toxin (3, 3.3, 3.6 and 4 μg/100 mg of body weight) are resuspended (in a volume not exceeding 5 μl) in water are injected in the other four groups. Each animal is injected in the last underside segment, using a 10 μL HamiltonTM syringe. The survival ratio is assessed within 24 h.

In order to test the activity of the refolded toxin six animals are injected with 5 μg each, using the same conditions as above.

### SUMMARY OF RESULTS

The results are summarised in Table 1. Less than 5% of renatured Cn5 toxin can be prepared using refolding buffer alone. PPI-agarose gives a yield of about 10% soluble protein, but it is mainly aggregated. DsbA-agarose gives a 10-15% yield of soluble protein, only 30% of which is monodispersed. The binary refolding matrix of mini-chaperone and DsbA gives a high yield of protein, of which 74% is monodisperse and 100% biologically active, as well as having the spectra of native toxin. The ternary matrix gives a 98% yield of soluble protein of which 89% is monodisperse and >100% biologically active and with native spectra. It is particularly interesting to note that the ternary matrix leads to the recovery of soluble protein having greater activity than the untreated native protein. ps Legend for Table 1

⁰NHS-activated Sepharose 4-fast flow.

¹100 mM potassium phosphate pH 8. 0.25 M L-arginine, 1 mM GSSG, 1 mMGSH, 2 mM EDTA.

²Mixed bed columns of mini-chaperone-agarose and DsbA-agarose in equal molar ratio.

³Mixed bed columns of mini-chaperone-agarose+DsbA-agarose+PPI-agarose in equal molar ratio.

⁴Not determined

^{a} The protein remaining soluble was measured using molar absorptivity A₂₇₆=18 080 M⁻¹ cm⁻¹ and by Bradford assays.

^{b} Evaluated by gel filtration chromatography.

TABLE 1
Refolding of Toxin Cn5 (120 μg/100 μL gel)

| | % protein recovery^{a} | | % as monodisperse | % Activity recovery |
|---|---|---|---|---|
| Gel type⁰ | (soluble fraction) | % as aggregated protein^{b} | protein^{b} | (of soluble fraction) |
| Refolding buffer | 1-5 | — | — | — |
| alone¹ | | | | |
| PPI-agarose | 10-12 | >11 | <1 | — |
| DsbA-agarose | 10-15 | ˜10 | 5 | Nd⁴ |
| Minichaperone-agarose | 12 | 9 | 3 | Nd⁴ |
| Binary refolding | 95 | 25 | 70 | 100 |
| matrix² | | | | |
| (mini-chaperone/Dsba) | | | | |
| Ternary refolding | 98 | 11 | 87 | >100 |
| Matrix³ | | | | |
| (mini- | | | | |
| chaperone/DSBA/PPI) | | | | |

We claim:

1. A method for promoting the folding of a polypeptide which method comprises contacting the polypeptide with a molecular chaperone and a foldase, wherein the foldase and optionally the molecular chaperone are immobilised onto a solid phase support.

2. A method according to claim 1, wherein the polypeptide is an unfolded or misfolded polypeptide.

3. A method according to claim 2, wherein the polypeptide comprises a disulphide.

4. A method according to claim 1, wherein the molecular chaperone is a fragment of a molecular chaperone with chaperonin activity.

5. A method according to claim 4, wherein the molecular chaperone is a fragment of a hsp-60 chaperonin, selected from the group consisting of mammalian hsp-60 and GroEL, or a derivative thereof.

6. A method according to claim 5 wherein the fragment is a fragment of GroEL which does not have an Alanine residue at position 262 and/or an Isoleucine residue at position 267 of the sequence of intact GroEL.

7. A method according to claim 6, wherein the fragment of GroEL has a Leucine residue at position 262 and/or a Methionine residue at position 267 of the sequence of intact GroEL.

8. A method according to claim 5, wherein the molecular chaperone fragment comprises a region which is homologous to at least one of fragments 191-376, 191-345 and 191-335 of the sequence of intact GroEL.

9. A method according to claim 1, wherein the foldase is selected from the group consisting of thiol/disulphide oxidoreductases and peptidyl-prolyl isomerases.

10. A method according to claim 9, wherein the thiol/disulphide oxidoreductase is selected from the group consisting of E. coli DsbA and mammalian PDI, or a derivative thereof.

11. A method according to claim 9, wherein the peptidyl prolyl isomerase is selected from the group consisting of cyclophilin, parbulen, SurA and FK506 binding proteins.

12. A method according to claim 1 comprising contacting the polypeptide with a molecular chaperone and both a thiol/disulphide oxidoreductase and peptidyl-prolyl isomerase wherein the thiol/disulphide oxidoreductase and/or the peptidyl-prolyl isomerase and optionally the molecular chaperone are immobilised onto a solid phase support.

13. A method according to claim 1 wherein the solid phase support is agarose.

14. A solid phase support having immobilised thereon a foldase.

15. A solid phase support having immobilised thereon a molecular chaperone and a foldase.

16. A column packed at least in part with a solid phase support according to claim 14 or 15.

17. A method for immobilising a disulphide-containing peptide onto a solid phase support, comprising the steps of:
a) reducing the disulphide in the polypeptide with a reducing agent, and removing the reducing agent under conditions so as to prevent re-oxidation;
b) reversibly blocking the thiol groups of the polypeptide;
c) contacting the solid phase with the thiol-blocked polypeptide at a non-acidic pH;
d) blocking any remaining active groups and removing uncoupled polypeptide by washing; and
e) regenerating the thiol groups on the bound polypeptide.

18. A method according to claim 17, wherein step c) is carried out at a pH between 7.5 and 9.5.

19. A solid phase support according to claim 14 or 15, or a column according to claim 16, obtainable by a method according to claim 17 or 18.

20. A thiol/disulphide oxidoreductase immobilised on a solid phase support obtainable by a method according to claim 17 or 18.

21. A peptidyl prolyl isomerase immobilised on a solid phase support obtainable by a method according to claim 17 or 18.

22. Use of a molecular chaperone and a foldase for promoting the folding of a polypeptide wherein the foldase and optionally the molecular chaperone are immobilised on a solid phase support.

23. Use according to claim 22 wherein the molecular chaperone is a fragment of GroEL comprising a Leucine residue at position 262 and/or a Methionine residue at position 267 of the sequence of intact GroEL.

24. A composition comprising a combination of a molecular chaperone and a foldase wherein the molecular chaperone and the foldase are immobilised on a solid phase support.

25. A method for promoting the folding of a polypeptide which method comprises contacting the polypeptide with a molecular chaperone and a foldase wherein the molecular chaperone consists essentially of at least one of fragments 191-376, 191-345 and 191-335 of the sequence of intact GroEL, or a homologue thereof.

26. A method according to claim 25 wherein said fragment does not have an alanine residue at position 262 and/or an isoleucine residue at position 267 of the intact GroEL sequence.

27. A method according to claim 26, wherein said fragment has a leucine residue at position 262 and/or a methionine residue at position 267 of the intact GroEL sequence.

28. A method according to claim 25, wherein the foldase is selected from the group consisting of thiol/disulphide oxidoreductases and peptidyl-prolyl isomerases.

29. A method according to claim 28, wherein the thiol/disulphide oxidoreductase is selected from the group consisting of E. coli DsbA and mammalian PDI, or a derivative thereof.

30. A method according to claim 28, wherein the peptidyl-prolyl isomerase is selected from the group consisting of cyclophilin, parbulen, SurA and FK506 binding proteins.

31. A composition comprising a combination of a molecular chaperone and a foldase wherein the molecular chaperone consists essentially of at least one of fragments 191-376, 191-345 and 191-335 of the sequence of intact GroEL, or a homologue thereof.

32. A polypeptide obtained by the method of the invention said polypeptide having greater than 100% of the biological activity of the corresponding native protein.

33. A polypeptide according to claim 32 which is a toxin.

34. A polypeptide according to claim 33 which is a scorpion toxin.
